(19) United States
(12) Patent Application Publication
(10) Pub. No.: US 2021/0290091 A1
(43) Pub. Date: Sep. 23, 2021

(54) METHOD AND SYSTEM FOR WELLNESS ESTIMATION OF A USER USING PULSE HARMONICS FROM PPG SIGNALS

(71) Applicant: Tata Consultancy Services Limited, Mumbai (IN)

(72) Inventors: Ramesh Kumar RAMAKRISHNAN, Bangalore (IN); Rahul Dasharath GAVAS, Bangalore (IN); Venkata Subramanian VIRARAGHAVAN, Bangalore (IN); Arpan PAL, Kolkata (IN); Balamuralidhar PURUSHOTHAMAN, Bangalore (IN); Lalit Kumar HISSARIA, Bangalore (IN)

(73) Assignee: Tata Consultancy Services Limited, Mumbai (IN)

(21) Appl. No.: 17/204,090

(22) Filed: Mar. 17, 2021

(30) Foreign Application Priority Data
Mar. 20, 2020 (IN) 202021012087

(51) Int. Cl. A61B 5/024; A61B 5/00; G16H 50/30

(52) U.S. Cl. CPC A61B 5/02416; G16H 50/30; A61B 5/7257

(57) ABSTRACT

Wellness estimation allows tracking of various health parameters and estimating health condition(s) of the user being monitored. Photophlethysmogram (PPG) based health monitoring systems exist. This disclosure relates generally to PPG based wellness monitoring, and more specifically to a pulse harmonics based wellness estimation. The system collects PPG signals from a user being monitored, as input. The system calculates 12 pulse harmonics from a fundamental frequency of the PPG signal. From the pulse harmonics, further a plurality of key features are extracted, and in turn a wellness metric and a wellness index are calculated, which represents health of the user.

### PRIORITY CLAIM

This U.S. patent application claims priority under 35 U.S.C. § 119 to: India Application No. 202021012087, filed on Mar. 20, 2020. The entire contents of the aforementioned application are incorporated herein by reference.

### TECHNICAL FIELD

This disclosure relates generally to wellness monitoring, and more particularly to a method and system for wellness estimation of a user.

### BACKGROUND

‘Wellness monitoring’ broadly refers to an activity of monitoring one or more health parameters and in turn health conditions of users. Depends on purpose/type of wellness monitoring, appropriate methods/systems are used. For example, if a person is being monitored for diagnosis of heart diseases, then suitable sensors for collecting and processing cardiac signals are used. Photophlethysmogram (PPG) is an optically obtained phlethysmogram that can be used to detect blood volume changes in microvascular bed of tissue. The PPG signals collected can be processed further, so as to perform the health estimation.

The inventors here have recognized several technical problems with such conventional systems, as explained below. State of the art systems that use PPG based health estimation approaches exist. However, PPG as a signal has different characteristics/components. As a result, different signal processing approaches can be adopted, each providing varying accuracy and convenience in terms of the signal processing and the health estimation. For example, one of the PPG signal processing approaches that is widely used is Heart Rate Variability (HRV) estimation. HRV consists of changes in time intervals between consecutive heartbeats. The HRV information estimated from PPG signals can be representative of various health conditions of the user. Such methods currently available provide measurement estimates of specific health parameters like heart rate, peripheral capillary oxygen saturation level (SPO2), Heart Rate Variability (HRV) features and not a holistic perspective of wellness. The user has to be well educated on how to interpret these measurements in relation to health and usually requires comparison of data over long periods.

### SUMMARY

Embodiments of the present disclosure present technological improvements as solutions to one or more of the above-mentioned technical problems recognized by the inventors in conventional systems. For example, in one embodiment, a processor implemented method for estimation of wellness of a user is provided. Initially a photoplethysmogram (PPG) signal from the user is collected, over a period of time, via one or more hardware processors. Further, a power spectral analysis of the PPG signal is performed using Fast Fourier Transforms (FFT) to generate a power spectrum of the PPG signal, via the one or more hardware processors. A fundamental frequency (f1) is then estimated as a frequency component with highest magnitude in the power spectrum of the PPG signal, via the one or more hardware processors. Further, 12 harmonics of the fundamental frequency as equal to multiples of the fundamental frequency for 12 iterations are calculated via the one or more hardware processors, and then power of each of the harmonics is calculated via the one or more hardware processors. A plurality of key distinguishing features from the calculated power of each of the 12 harmonics are determined via the one or more hardware processors. Further, a wellness metric is calculated based on the plurality of key distinguishing features, via the one or more hardware processors, wherein the wellness metric represents health condition of the user.

In another embodiment, a system for estimation of the wellness of a user is provided. The system includes one or more hardware processors, one or more communication interfaces, and one or more memory (102) storing a plurality of instructions. The plurality of instructions when executed cause the one or more hardware processors to collect a photoplethysmogram (PPG) signal from the user, over a period of time. The system then performs a power spectral analysis of the PPG signal is performed using Fast Fourier Transforms (FFT) to generate a power spectrum of the PPG signal, via the one or more hardware processors. The system further estimates a fundamental frequency (f1) is then estimated as a frequency component with highest magnitude in the power spectrum of the PPG signal, via the one or more hardware processors. Further, 12 harmonics of the fundamental frequency as equal to multiples of the fundamental frequency for 12 iterations are calculated by the system via the one or more hardware processors, and then power of each of the harmonics is calculated via the one or more hardware processors. The system then determines a plurality of key distinguishing features from the calculated power of each of the 12 harmonics via the one or more hardware processors. Further, a wellness metric is calculated based on the plurality of key distinguishing features, via the one or more hardware processors, wherein the wellness metric represents health condition of the user.

In yet another embodiment, a non-transitory computer readable medium for estimation of the wellness of a user is provided. The non-transitory computer readable medium initially collects a photoplethysmogram (PPG) signal from the user, over a period of time, via one or more hardware processors. Further, a power spectral analysis of the PPG signal is performed using Fast Fourier Transforms (FFT) to generate a power spectrum of the PPG signal, via the one or more hardware processors. A fundamental frequency (f1) is then estimated as a frequency component with highest magnitude in the power spectrum of the PPG signal, via the one or more hardware processors. Further, 12 harmonics of the fundamental frequency as equal to multiples of the fundamental frequency for 12 iterations are calculated via the one or more hardware processors, and then power of each of the harmonics is calculated via the one or more hardware processors. A plurality of key distinguishing features from the calculated power of each of the harmonics are determined via the one or more hardware processors. Further, a wellness metric is calculated based on the plurality of key distinguishing features, via the one or more hardware processors, wherein the wellness metric represents health condition of the user.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates an exemplary system for wellness estimation of a user using pulse harmonics extracted from PPG signal of the user, according to some embodiments of the present disclosure.

FIG. 2 is a flow diagram depicting steps involved in the process of the pulse harmonics based health estimation of the user, using the system of FIG. 1, according to some embodiments of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments. It is intended that the following detailed description be considered as exemplary only, with the true scope being indicated by the following claims.

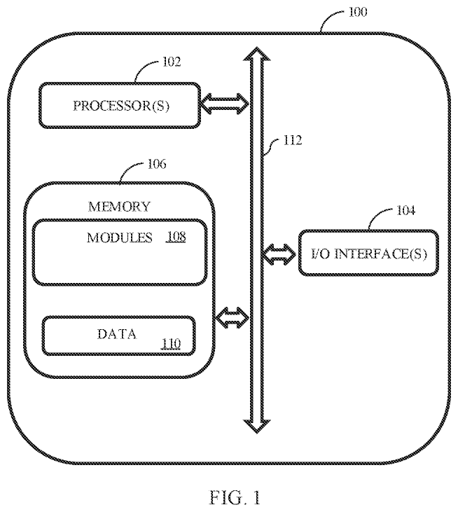

FIG. 1 illustrates an exemplary system for wellness estimation of a user using pulse harmonics extracted from PPG signal of the user, according to some embodiments of the present disclosure. The system 100 may be implemented in a computing device. Examples of the computing device include, but are not limited to, mainframe computers, workstations, personal computers, desktop computers, minicomputers, servers, multiprocessor systems, laptops, a cellular communicating device, such as a personal digital assistant, a smart phone, and a mobile phone; and the like. The system 100, implemented using the computing device, includes one or more hardware processor(s) 102, 10 interface(s) 104, and a memory 106 coupled to the processor 102. The processor 102 can be a single processing unit or a number of units. The hardware processor 102, the memory 106, and the 10 interface 104 may be coupled by a system bus such as a system bus 112 or a similar mechanism. The processor 102 may be implemented as one or more microprocessors, microcomputers, microcontrollers, digital signal processors, central processing units, state machines, logic circuitries, and/or any devices that manipulate signals based on operational instructions. Among other capabilities, the processor 102 is configured to fetch and execute computer-readable instructions and data stored in the memory 106.

Functions of the various elements shown in the figures, including any functional blocks labeled as “processor(s)”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non-volatile storage. Other hardware, conventional and/or customized, may also be included.

The 10 interfaces 104 may include a variety of software and hardware interfaces, for example, interface for peripheral device(s), such as a keyboard, a mouse, an external memory, and a printer. Further, the IO interfaces 104 may enable the computing device to communicate with other computing devices, such as a personal computer, a laptop, and like.

The memory 106 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes. The memory 106 may also include module(s) 108 and data 110.

The modules 108 may include routines, programs, objects, components, data structures, and so on, which perform particular tasks or implement particular abstract data types. The modules 108 may include programs or computer-readable instructions or coded instructions that supplement applications or functions performed by the system 100. The modules 108 may also be used as, signal processor(s), state machine(s), logic circuitries, and/or any other device or component that manipulates signals based on operational instructions. Further, the modules 108 can be used by hardware, by computer-readable instructions executed by the one or more hardware processors 102, or by a combination thereof. In an embodiment, the modules 108 can include various sub-modules (not shown), The other module(s) may include programs or coded instructions that supplement applications and functions of the computing device.

The data 110, amongst other things, serves as a repository for storing data processed, received, and generated by one or more of the module(s) 108. The data 110 includes, for example, history of PPG signals collected over a period of time, wellness metrics calculated for each set of input PPG signals collected, and any such data that are collected or generated during the wellness monitoring being performed by the system 100. The other data includes data generated as a result of the execution of one or more modules in the other module(s).

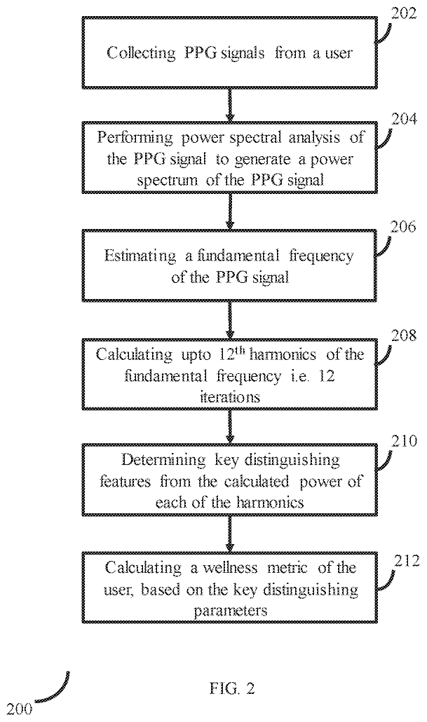

Steps involved in the process of performing the wellness estimation by the system 100 are depicted in FIG. 2, and are explained with reference to the system 100.

FIG. 2 is a flow diagram depicting steps involved in the process of the pulse harmonics based wellness estimation of the user, using the system of FIG. 1, according to some embodiments of the present disclosure. Initially the system 100 collects (202) a PPG signal from a user (who may be a patient being monitored), using any known sensor such as a pulse oximeter. The PPG signal maybe collected for a specific time period as may be required.

Further, the system 100 performs (204) a power spectral analysis of the collected PPG signal. In an embodiment, the system 100 may process the collected PPG signal at once, or may split the PPG signal to segments of specific length and process, as configured. The system may use Fast Fourier Transform (FFT) or any other such suitable approach to perform the power spectral analysis. After performing the power spectral analysis, the system 100 may use direct component of the power spectrum signals for A3 and A4 metrics.

The system 100 further estimates (206) a frequency component with highest magnitude in the power spectrum of the PPG signal, and which is closest to a normal heart rate, as a fundamental frequency (f1) of the PPG signal. Here the value of the ‘normal heart rate’ may be pre-configured with the system 100.

The system 100 further calculates n^{th} harmonics (pulse harmonics) of the fundamental frequency (f1) as multiples/iterations of the fundamental frequency (f1) i.e.

n^{th} harmonics fₙ=n*f1 (1)

where f1 is the fundamental frequency, and n=2, 3, . . . , 12

The system 100 further calculates power (pn) of each of the calculated harmonics, and the calculated power is denoted as p2, p3, . . . p12. It was observed that peaks are too small to be processed, post 12^{th} harmonic. However, it is to be noted that the system 100 can be configured to calculate harmonics and power spectrum past the 12^{th} harmonics if required.

Further at step 210, the system 100 determines (210) a plurality of key distinguishing features from the calculated power of each of the harmonics, by computing a separation capability of each of the harmonic powers independently of pulse harmonics p2, p3, . . . p12 so as to determine one or more of the features p2, p3, . . . p12 as key distinguishing features relating to variations in health condition, which may be result of any fitness activity being performed by the user. The ‘separation capability’ refers to ability of a feature to consistently distinguish between different states of a ground truth. The ground truth in the case of wellness estimation is a user being monitored being healthy or unhealthy.

Based on the determined key distinguishing features, the system 100 calculates (212) a wellness metric. The wellness metric includes one or more wellness indexes that represent different health conditions of the user being monitored. In order to determine improvement in health condition of the user over a period of time, wellness metrices are calculated at a first time instance T1 and at a second time instance T2. The wellness metric at time T1 and the wellness metric at time T2 are compared to generate a first wellness index (A1), a second wellness index (A2), a third wellness index (A3), and a fourth wellness index (A4).

The first wellness index (A1) of the user is determined as:

$${A}_{1} = \frac{[ α ( T 1 ) - α ( T 2 ) ]}{α ( T 1 )} - ( 2 )$$

where, α=the sum of the powers of the 5^{th} to 12^{th} harmonics from among the 12 iterations, T₁ is the first time instance, and T₂ is the second time instance.

The first wellness index (A1) represents a general health condition of the user being monitored.

The second wellness index (A2) of the user is determined as:

$${A}_{2} = \frac{[ α ( T 1 ) - α ( T 2 ) ]}{α ( T 1 )} - ( 2 )$$

where, α=the sum of the powers of the 7^{th} 9^{th} and 10^{th} harmonics from among the 12 iterations, T₁ is the first time instance, and T₂ is the second time instance.

The second wellness index (A2) represents improvement in health of the user due to one or more fitness activities performed during the time between T1 and T2 a general health condition of the user being monitored.

The third wellness index (A3) is determined as:

$$A 3 = \frac{{\sum }_{i = 5}^{12} {P}_{i}}{{\sum }_{i = 1}^{12} {P}_{i}}$$

where P is power of the i^{th} harmonic.

The third wellness index A3 represents a point in time general wellness of the user.

The fourth wellness index (A₄) is determined as:

$$A 4 = \frac{{\sum }_{i = 7 , 9 , 10} {P}_{i}}{{\sum }_{i = 1}^{12} {P}_{i}}$$

where P being the power of the i^{th} harmonic.

The fourth wellness index A4 represents a point in time wellness of the user due to effect of the one or more physical activities.

In a practical application, the system 100 can be configured to determine improvement in wellness or health condition of a user, over a period of time, by applying the method 200. In this approach, for a user, the system 100 calculates a first wellness metric at a first time instance T1, and a second wellness metric at a second time instance T2. The system 100 then compares the first wellness metric and the second wellness metric. By virtue of the comparison, the system 100 generates a first wellness index A1 a second wellness index A2. A1 represents improvement in a general wellness of the user, and A2 represents improvement in health of the user, due to one or more physical activities performed by the user during the time period between T1 and T2. For example, consider that the user had been practicing Yoga during this time period. In that case the wellness index A2 represents improvement in health condition of the user due to Yoga.

In an experimental setup, a first group of people (referred to as Yoga Group) containing 36 subjects practiced yoga for an experiment period of 12 days. PPG signals were taken from each of the subjects on first day of the experiment period and on last day (i.e. 12^{th} day) of the experiment period. A second group of people (referred to as a ‘control group’) containing 24 subjects, with the subjects having different exercise patterns. From the subjects in the control group also, PPG signals were collected on first day and on the 12^{th} day of the experiment period. Table. 1 below contains values of different parameters for the subjects in the first group and the second group.

TABLE 1
Pulse Harmonics

| Feature | Yoga group | Control group |
|---|---|---|
| f1 | 0.22 | 0.31 |
| P1 | 0.37 | 0.26 |
| P2 | 0.16 | 0.57 |
| P3 | 0.65 | 0.29 |
| P4 | 0.16 | 0.54 |
| P5 | 0.01 | 0.47 |
| P6 | 0.03 | 0.45 |
| P7 | 0.01 | 0.84 |
| P8 | 0.01 | 0.54 |
| P9 | 0.0001 | 0.83 |
| P10 | 0.01 | 0.73 |
| P11 | 0.02 | 0.41 |
| P12 | 0.01 | 0.27 |

The values indicate that while there has not been any significant change in the power-values for the subjects in the control group, there is a significant change for the subjects in the yoga group, from P5 to P12, which in turn depicts improvement in wellness/health of the subjects due effect of the physical activity (which in this example is Yoga).

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional budding blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A processor implemented method for estimation of wellness of a user, comprising:
collecting a photoplethysmogram (PPG) signal from the user, over a period of time, via one or more hardware processors;
performing a power spectral analysis of the PPG signal using Fast Fourier Transforms (FFT) to generate a power spectrum of the PPG signal, via the one or more hardware processors;
estimating a fundamental frequency (f1) as a frequency component with highest magnitude in the power spectrum of the PPG signal, via the one or more hardware processors;
calculating harmonics of the fundamental frequency as equal to multiples of the fundamental frequency for 12 iterations to generate 12 harmonics, via the one or more hardware processors;
calculating power of each of the 12 harmonics, via the one or more hardware processors;
determining a plurality of key distinguishing features from the calculated power of each of the 12 harmonics, via the one or more hardware processors; and
calculating a wellness metric based on the plurality of key distinguishing features, via the one or more hardware processors, wherein the wellness metric represents health condition of the user.

2. The method as claimed in claim 1, wherein the plurality of key distinguishing features are computed by calculating a separation capability of the power of each of the harmonics independently.

3. The method as claimed in claim 1, wherein the wellness metric is used to determine improvement in health condition of the user over a period of time, comprising:
calculating a first wellness metric at a first time instance (T1), via the one or more hardware processors;
calculating a second wellness metric at a second time instance (T2), via the one or more hardware processors; and
comparing the first wellness metric and the second wellness metric, comprising:
determining a first wellness index (A1) of the user, wherein the first wellness index represents a general wellness of the user;
determining a second wellness index (A2) of the user, wherein the second wellness index represents improvement in health of the user due to one or more physical activities performed during the time between T1 and T2;
determining a third wellness index (A3) of the user, wherein the third wellness index represents a point in time general wellness of the user; and
determining a fourth wellness index (A4) of the user, wherein the fourth wellness index represents a point in time wellness of the user due to effect of the one or more physical activities.

4. The method as claimed in claim 3, wherein the first wellness index (A₁) is determined as:
$${A}_{1} = \frac{[ α ( T 1 ) - α ( T 2 ]}{α ( T 1 )}$$
where, α=the sum of the powers of the 5^{th} to 12^{th} harmonics from among the 12 iterations, T1 is the first time instance, and T2 is the second time instance.

5. The method as claimed in claim 3, wherein the second wellness index (A₂) is determined as:
$${A}_{2} = \frac{[ α ( T 1 ) - α ( T 2 ) ]}{α ( T 1 )}$$
where, α=the sum of the powers of the 7^{th}, 9^{th} and 10^{th} harmonics from among the 12 iterations, T1 is the first time instance, and T2 is the second time instance.

6. The method as claimed in claim 3, wherein the third wellness index (A₃) is determined as:
$$A 3 = \frac{{\sum }_{i = 5}^{12} {P}_{i}}{{\sum }_{i = 1}^{12} {P}_{i}}$$
where P is power of the i^{th} harmonic.

7. The method as claimed in claim 3, wherein the fourth wellness index (A₄) is determined as:
$$A 4 = \frac{{\sum }_{i = 7 , 9 , 10} {P}_{i}}{{\sum }_{i = 1}^{12} {P}_{i}}$$
where P being the power of the i^{th} harmonic.

8. A system for estimation of wellness of a user, comprising:
one or more hardware processors;
one or more communication interfaces; and
one or more memory storing a plurality of instructions, wherein the plurality of instructions when executed cause the one or more hardware processors to:
collect a photoplethysmogram (PPG) signal from the user, over a period of time;
perform a power spectral analysis of the PPG signal using Fast Fourier Transforms (FFT) to generate a power spectrum of the PPG signal;
estimate a fundamental frequency (f1) as a frequency component with highest magnitude in the power spectrum of the PPG signal, via the one or more hardware processors;
calculate harmonics of the fundamental frequency as equal to multiples of the fundamental frequency for 12 iterations to generate 12 harmonics;
calculate power of each of the 12 harmonics;
determine a plurality of key distinguishing features from the calculated power of each of the 12 harmonics;
calculate a wellness metric based on the plurality of key distinguishing features, wherein the wellness metric represents health condition of the user.

9. The system as claimed in claim 8, wherein the system computes the plurality of key distinguishing features by calculating a separation capability of the power of each of the harmonics independently.

10. The system as claimed in claim 8, wherein the system uses the wellness metric to determine improvement n health condition of the user over a period of time, by:
calculating a first wellness metric at a first time instance (T1), via the one or more hardware processors;
calculating a second wellness metric at a second time instance (T2), via the one or more hardware processors; and
comparing the first wellness metric and the second wellness metric, comprising:
determining a first wellness index (A₁) of the user, wherein the first wellness index represents a general wellness of the user;
determining a second wellness index (A₂) of the user, wherein the second wellness index represents improvement in health of the user due to one or more physical activities performed during the time between T1 and T2;
determining a third wellness index (A₃) of the user, wherein the third wellness index represents a point in time general wellness of the user; and
determining a fourth wellness index (A₄) of the user, wherein the fourth wellness index represents a point in time wellness of the user due to effect of the one or more physical activities.

11. The system as claimed in claim 10, wherein the system determines the first wellness index (A₁) as:
$${A}_{1} = \frac{[ α ( T 1 ) - α ( T 2 ) ]}{α ( T 1 )}$$
where, α=the sum of the powers of the 5^{th} to 12^{th} harmonics from among the 12 iterations, T₁ is the first time instance, and T₂ is the second time instance.

12. The system as claimed in claim 10, wherein the system determines the second wellness index (A₂) as:
$${A}_{2} = \frac{[ α ( T 1 ) - α ( T 2 ) ]}{α ( T 1 )}$$
where, α=the sum of the powers of the 7^{th}, 9^{th} and 10^{th} harmonics from among the 12 iterations, T₁ is the first time instance, and T₂ is the second time instance.

13. The system as claimed in claim 10, wherein the system determines the third wellness index (A₃) as:
$$A 3 = \frac{{\sum }_{i = 5}^{12} {P}_{i}}{{\sum }_{i = 1}^{12} {P}_{i}}$$
where P is power of the i^{th} harmonic.

14. The system as claimed in claim 10, wherein the system determines a fourth wellness index (A₄) is determined as:
$$A 4 = \frac{{\sum }_{i = 7 , 9 , 10} {P}_{i}}{{\sum }_{i = 1}^{12} {P}_{i}}$$
where P being the power of the i^{th} harmonic.

15. A computer program product comprising a non-transitory computer readable medium having a computer readable instructions embodied therein, wherein the computer readable instructions, when executed, cause to perform estimation of a user, by:
collecting a photoplethysmogram (PPG) signal from the user, over a period of time, via one or more hardware processors;
performing a power spectral analysis of the PPG signal using Fast Fourier Transforms (FFT) to generate a power spectrum of the PPG signal, via the one or more hardware processors;
estimating a fundamental frequency (f1) as a frequency component with highest magnitude in the power spectrum of the PPG signal, via the one or more hardware processors;
calculating harmonics of the fundamental frequency as equal to multiples of the fundamental frequency for 12 iterations to generate 12 harmonics, via the one or more hardware processors;
calculating power of each of the 12 harmonics, via the one or more hardware processors;
determining a plurality of key distinguishing features from the calculated power of each of the 12 harmonics, via the one or more hardware processors; and
calculating a wellness metric based on the plurality of key distinguishing features, via the one or more hardware processors, wherein the wellness metric represents health condition of the user.
